(19) United States
(12) Patent Application Publication
(10) Pub. No.: US 2021/0113485 A1
(43) Pub. Date: Apr. 22, 2021

(54) COMPOSITE FIBERS AND MATRICES THEREOF

(71) Applicant: UNIVERSITY OF CONNECTICUT, Farmington, CT (US)

(72) Inventors: Sangamesh G. KUMBAR, Fannington, CT (US); Matthew David HARMON, Fannington, CT (US)

(21) Appl. No.: 17/006,200

(22) Filed: Aug. 28, 2020

Related U.S. Application Data
(63) Continuation-in-part of application No. 15/536,289, filed on Jun. 15, 2017.
(60) Provisional application No. 62/109,238, filed on Jan. 29, 2015.

(51) Int. Cl. A61K 9/70; C08L 67/04; C12N 5/077; D01F 2/28; D01F 6/84

(52) U.S. Cl. CPC A61K 9/70; C08L 67/04; C12N 5/0655; D01F 2/28; C12N 2533/78; C08L 2203/12; C08L 2205/16; C08L 2203/02; C12N 2533/40; D01F 6/84

(57) ABSTRACT

Composite fibers, such as poly(lactic-co-glycolic acid) (PLGA)-cellulose acetate (CA) composite fibers, matrices including such fibers, and methods for making and using such fibers and matrices are disclosed.

### CROSS-REFERENCE

This application is a continuation in part of U.S. application Ser. No. 15/536,289 filed Jun. 15, 2017, which is a national filing of PCT/US2016/15104 filed Jan. 27, 2016, which claims support to U.S. Provisional Patent Application Ser. No. 62/109,238 filed Jan. 29, 2015, incorporated by reference herein in its entirety.

### STATEMENT OF GOVERNMENT SUPPORT

This invention was made with government support under Grants #IIP-1311907, IIP-13555327, and EFRI-1332329 awarded by the National Science Foundation. The government has certain rights in the invention.

### BACKGROUND

The loss of physical and mechanical characteristics of electrospun polyester matrices, such as Poly(lactic-co-glycolic acid) (PLGA), has not been addressed previously. Shrinkage of PLGA electrospun fiber matrices and associated changes with, morphology, degradation and mechanical properties have been ignored. Alternative approaches based on polycaprolactone (PCL) and copolymers have been ineffective, as the degradation rate of PCL based fiber matrices is generally slow and is dependent on the polymer composition and initial molecular weight. These alternative scaffold materials lack in the desired degradation rate and may not be suitable for transient biomedical applications.

### SUMMARY OF THE INVENTION

In a first aspect, the invention provides fibers, comprising polyester and a cellulose derivative, wherein the fiber has a diameter of less than about 5000 nm. In one embodiment, the cellulose derivative is selected from the group consisting of cellulose acetate (CA), ethyl cellulose, ethyl hydroxymethylcellulose, and combinations thereof. In a specific embodiment, the cellulose derivative comprises CA. In a further embodiment, the polyester is selected from the group consisting of polylactic acid (PLA), polyglycolic acid (PGA), Poly(lactic-co-glycolic acid) (PLGA), poly(caprolactone) (PCL), Polyhydroxyalkanoate (PHA); Polyhydroxybutyrate (PHB); Polyethylene adipate (PEA); Polybutylene succinate (PBS); Poly(3-hydroxybutyrate-co-3-hydroxyvalerate)(PHBV); Polyethylene terephthalate (PET); Polybutylene terephthalate (PBT); Polytrimethylene terephthalate (PTT); Polyethylene naphthalate (PEN), or combinations thereof. In other embodiments, the polyester is selected from the group consisting of PLA, PGA, PLGA, PCL and combinations thereof. In a specific embodiment, the polyester comprises PLGA. In a further embodiment, the polyester is at least 70% by weight of the fiber. In another embodiment, wherein the cellulose derivative is present in the fiber at between about 3% to about 50% weight percentage of polyester in the fiber. In one embodiment, the cellulose derivative comprises CA, and wherein the CA is present in the fiber at between about 3% to about 25%, or between about 5% to about 15% weight percentage of polyester in the fiber.

In another aspect, the invention provides matrices comprising a plurality of the nanofibers of any one of claims 1-17, wherein the nanofibers are linked together to form the matrix. In a further aspect, the invention provides methods for making fibers, comprising:

(a) adding a cellulose derivative, such as CA, to a solution of a polyester dissolved in an organic solvent to form a cellulose derivative-polyester solution; and

(b) electrospinning the cellulose derivative-polyester solution to form a cellulose derivative-polyester fiber.

### DESCRIPTION OF THE FIGURES

FIG. 1A-D. Gross morphology of electrospun neat PLGA (A, B) and with 10% doping of CA (C, D) before (A, C) and after seven days (B, D) of incubation at 37° C. in PBS (n=3).

FIG. 2A-E. Morphology of fiber matrices doped with varying amount of CA following incubation in PBS at 37° C. for tensile testing. All these matrices were cut into dog-bone shapes measuring 5×10 mm (1:2 ratio). A through E represent increasing amounts of cellulose acetate doping, 0% to 20%. (A—PLGA neat, B—with 5% CA, C—with 10% CA, D—with 15% CA, E—with 20% CA).

FIG. 3A-F. Cell survival and morphology of human articular chondrocytes seeded on neat electrospun PLGA and 15% CA doped fiber matrices were determined by viability/cytotoxicity assay. Live cells appear as fluorescent green color and dead cells as fluorescent red. A noticeable change in cellular morphology and proliferative performance is seen. (Images A, B, D, and E—are 20×. Images C & F are 10×.)

FIG. 4A-D. SEM micrographs of human articular chondrocytes seeded fiber matrices at a time point of 3 (A & C) and 7 (B & D) days on neat PLGA (A & B) and 15% CA (C & D) doped fiber matrices.

FIG. 5. Relative tensile modulus (stress vs. strain) results of cellulose acetate doped PLGA scaffolds after dry and wet (37° C. buffered media) incubation.

FIG. 6A-C. Confocal images showing the feasibility of uniform model protein functionalization on the fiber matrix PLGA: CA (80:20) with BSA. For visualization FITC conjugated BSA (BSA-FITC) was chemically tethered to fiber matrix via hydroxyl groups present on CA backbone in the nanofibers. Prior to imaging matrices were washed repeatedly with DI water to remove any adsorbed protein and incubated in PBS for 2 hours. These images A-C were taken at the different locations of the matrix show uniformity of the matrix. Magnification 10×.

### DETAILED DESCRIPTION OF THE INVENTION

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

As used herein, “about” means+/−5% of the recited parameter.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In a first aspect, the present invention provides fibers, comprising polyester and a cellulose derivative, wherein the fiber has a diameter of less than about 5000 nm. As used herein, a “fiber” is a solid that has a length significantly greater than its width. As disclosed in the examples that follow, the cellulose derivative improves hydrophilicity of the polyester, with particular benefits for use of the fibers in, for example, soft and hard tissue regeneration, skin applications such as wound healing, and biomedical applications. The fibers of the invention can be made by any suitable process, including but not limited to electrospinning as disclosed in the examples.

In various embodiments, the fibers may have a diameter between about 10 nm and about 5000 nm, about 10 nm and about 2500 nm, about 10 nm and about 1000 nm, about 10 nm and about 250 nm, about 10 and about 100 nm, about 50 nm and about 5000 nm, about 50 nm and about 2500 nm, about 50 nm and about 1000 nm, about 50 nm and about 250 nm, about 50 and about 100 nm, about 100 nm and about 5000 nm, about 100 nm and about 2500 nm, or about 100 nm and about 1000 nm.

The length of the fibers may be any suitable length. In one embodiment, the fiber length may be between about 2 cm and about 60 cm in length. In various further embodiments, the length is between about 2 cm and about 58 cm, about 2 cm and about 55 cm, about 2 cm and about 52 cm, about 2 cm and about 50 cm, about 5 cm and about 60 cm, about 5 cm and about 58 cm, about 5 cm and about 55 cm, about 5 cm and about 52 cm, about 5 cm and about 50 cm, about 10 cm and about 60 cm, about 10 cm and about 58 cm, about 10 cm and about 55 cm, about 10 cm and about 52 cm, or about 10 cm and about 50 cm in length. In one specific embodiment, the fiber length is between about 10 cm and 50 cm in length, which is particularly useful in the preparation of matrices for implantation.

The fibers may comprise any suitable cellulose derivative such as cellulose esters and ethers. In exemplary embodiments, the cellulose derivative comprises cellulose acetate (CA), ethyl cellulose, ethyl hydroxymethylcellulose, or combinations thereof. In one particular embodiment, the cellulose derivative comprises CA. As shown in the examples that follow, CA addition (as an example of a cellulose derivative) improves the hydrophilicity, and avoids rapid shrinkage and increased brittleness, of polyester fibers (such as PLGA) that limit the use of matrices of such polyester fibers in aqueous environments, such as physiological conditions.

In one embodiment, the molecular weight of the CA in the fiber is between about 20,000 D and about 75,000 D. In various further embodiments, the CA molecular weight is between about 30,000 D and about 50,000 D, or about 50,000 D. As will be understood by those of skill in the art, use of higher molecular weight CA requires less weight of CA with polyester to produce fibers of similar viscosity to those made with lower molecular weight CA. Thus, the molecular weight of CA present in the fibers can be modified for intended fiber characteristics.

Any suitable polyester may be used in the fibers of the invention. In various embodiments, the polyester is selected from the group consisting of polylactic acid (PLA), polyglycolic acid (PGA), Poly(lactic-co-glycolic acid) (PLGA), poly(caprolactone) (PCL), Polyhydroxyalkanoate (PHA); Polyhydroxybutyrate (PHB); Polyethylene adipate (PEA); Polybutylene succinate (PBS); Poly(3-hydroxybutyrate-co-3-hydroxyvalerate)(PHBV); Polyethylene terephthalate (PET); Polybutylene terephthalate (PBT); Polytrimethylene terephthalate (PTT); Polyethylene naphthalate (PEN); or combinations thereof. In one specific embodiment, the polyester is selected from the group consisting of PLA, PGA, PLGA, PCL) and combinations thereof. In a particular embodiment, the polyester comprises PLGA. The term “PLGA” refers to poly(lactic-co-glycolic acid) that is synthesized by means of random ring-opening co-polymerization of two different monomers, the cyclic dimers (1,4-dioxane-2,5-diones) of glycolic acid and lactic acid. Depending on the ratio of lactide to glycolide used for the polymerization, different forms of PLGA can be obtained: these are usually identified in regard to the monomers' ratio used (e.g. PLGA 50:50 identifies a copolymer whose composition is 50% PLA and 50% PGA). Commercially PLGA is available in various ratios such as 50:50, 65:35 and 85:15. In one specific embodiment, the PLGA comprises PLA at 50% or less, to maximize desired flexibility of the fibers. In various embodiments, the PLGA is PLGA 50:50, PLGA 45:55, PLGA 40:60, PLGA 35:65, PLGA 30:70, or PLGA 25:75. Any suitable molecular weight of PLGA can be used in the fibers. In one embodiment, the PLGA molecular weight ranges between about 40 kD to about 250 kD. In various other embodiments, the PLGA molecular weight is between about 40 kD to about 225 kD, about 40 kD to about 200 kD, about 40 kD to about 175 kD, about 40 kD to about 150 kD, about 40 kD to about 125 kD, about 40 kD to about 100 kD, about 40 kD to about 75 kD, or about 40 kD to about 50 kD.

In one embodiment, the polyester is at least 70% by weight of the fiber. In various further embodiments, the polyester is at least 75%, 80%, 85%, 90%, 91%, 92%, 93%, 94%, 95%, or more by weight of the fiber.

In one embodiment, the cellulose derivative is present in the fiber at between about 3% to about 50% weight percentage of polyester in the fiber. In various other embodiments, the cellulose derivative is present in the fiber at between about 3% to about 40%, about 3% to about 35%, about 3% to about 30%, about 3% to about 25%, about 3% to about 20%, about 3% to about 15%, about 3% to about 10%, about 5% to about 50%, about 5% to about 40%, about 5% to about 35%, about 5% to about 30%, about 5% to about 25%, about 5% to about 20%, about 5% to about 15%, about 5% to about 10%, about 10% to about 50%, about 10% to about 40%, about 10% to about 35%, about 10% to about 30%, about 10% to about 25%, about 10% to about 20%, or about 10% to about 15% weight percentage of polyester in the fiber.

In one particular example, the cellulose derivative comprises CA, and the CA is present in the fiber at between about 3% to about 25% weight percentage of polyester in the fiber. In various further embodiments, the CA is present in the fiber at between about 3% to about 20%, about 3% to about 15%, about 3% to about 10%, about 5% to about 25%, about 5% to about 20%, about 5% to about 15%, about 5% to about 10% weight percentage of polyester in the fiber. The inventors have discovered that addition to the polyester fiber of about 3% to about 5% of CA significantly increases the fiber hydrophilicity. In particular embodiments, the polyester is selected from the group consisting of PLA, PGA, PLGA, PCL and combinations thereof. In a particular embodiment, the polyester comprises PLGA.

The inventors have discovered that properties of the resulting fibers (and matrices comprising the fibers) can be tuned by appropriate manipulation of the cellulose derivative molecular weight and concentration. By way of non-limiting example, use of 30 kD CA present in the fibers at between about 5% to about 15% weight percentage of polyester in the fiber results in matrices comprising the fibers having wettability and strength characteristics Suitable for repairing soft tissues such as skin, tendon, blood vessel, ligament, cardiac patch, skeletal muscle and nerve scaffolds. At higher CA ratios the matrices comprising the fibers are more of a fibrous gel that allows fluid retention and delivery bioactive molecules. Thus, the resulting matrices can perform all the functions of a hydrogel, but with an ordered structure. The matrix is not chemically or physically cross-linked as a hydrogel to stabilize its 3D structure. Cross-linked structures take longer time to degrade than the parent material and nature of cross-linking agents may have adverse effect on tissue compatibility. In general ordered fibrous structure will present higher tensile properties than the hydrogel more suitable for tissue regeneration applications. The fiber structure will guide the tissue regeneration along the length of the fiber orientation and void tissue infiltration. Often hydrogels fails to promote cell assignment, tissue ingrowth due to dense nature and steric hindrance offered by the matrix.

The fibers may comprise any additional components that may be suitable for an intended use of the fibers. In various embodiments, the fibers may comprise between about 1% to about 15% by weight of “cargo”, including but not limited to therapeutic agents, diagnostic agents, peptides, growth factors, detectable labels, etc. In one non-limiting embodiment, the fibers comprise at least 70% by weight of polyester (such as PLGA), between about 5% to about 25% by weight of cellulose derivative (such as CA), with some or all of the remainder comprising cargo such as that disclosed above. Matrices comprising such fibers are of particular for soft and hard tissue regeneration, skin applications such as wound healing, and biomedical applications, etc. The cargo can be incorporated into the fiber using standard chemical modification techniques to link the molecules to the polyester and/or the cellulose derivative in the fiber. For example, the availability of abundant —OH and —COOH groups in the derivatized cellulose (such as CA) in the fiber/resulting matrix provides uniform matrix functionalization, reproducibility, and improved matrix hydrophilicity. This is a significant improvement over prior polyester fibers/matrices. Prior physical encapsulation of bioactive molecules such as proteins, peptides and chemical drugs in polyester nanofiber matrices have resulted in a majority of the drug (70-90%) releases in the first 1-2 hours due to high surface area of the nanofiber matrix. Efforts to sustain the bioactive molecule include core-shell nanofiber fabrication or chemical tethering are tedious and results are not reproducible. In case of chemical tethering these polyesters needs to be modified first to create functional groups such as —OH or —COOH by a mild acid or base treatment which will affect the matrix integrality in terms of morphology, strength and reduction in matrix molecular weight.

In another aspect, the invention provides matrices comprising a plurality of the fibers of any embodiment or combination of embodiments of the invention. The matrices of the invention can be used, for example, in soft and hard tissue regeneration, skin applications such as wound healing, and biomedical applications, as described above and in the examples. In the dry state, the fibers interact via non-covalent bonding. Once placed in aqueous conditions (such as physiological conditions), hydroxyl moieties on the cellulose derivative participate in extensive inter- and intra-molecular hydrogen bonding. This results in insolubility of CA in aqueous environment and significantly improved matrix hydrophilicity, which increases with increasing percentage of the cellulose derivative in the fiber. Thus, the matrices are far less susceptible to shrinkage and deformation than previous polyester-based matrices, and provide, for example, significantly improved drug release profiles. In various embodiments, the fibers may be randomly distributed in the matrix, or may be parallel. The matrices may be a single layer, or may be present in multiple layers.

The matrices can be produced using any suitable technique, including but not limited to the electrospinning process disclosed herein. Based on the present disclosure, it is well within the level of one of skill in the art to make the matrices of the present invention. The matrices may be of any suitable size for an intended use. As will be understood by those of skill in the art, the matrix can be cut to any size from a larger sheet of, for example, electrospun matrix. When cut, fibers within the matrix may have varied lengths. In the case of random fibers placed within the matrix they can be of any size since a single fiber can have length more than 2 cm due to its winding placement within a matrix of even 50 mm². In the case of parallel fibers, fiber length may differ to the extent the matrix is cut. In one embodiment, the matrix may be between about 50 mm² and about 80 cm² in size. In various further embodiments, the size is between about 50 mm² and about 75 cm², about 50 mm² and about 70 cm², about 50 mm² and about 65 cm², about 50 mm² and about 60 cm², about 75 mm² and about 80 cm², about 75 mm² and about 75 cm², about 75 mm² and about 70 cm², about 75 mm² and about 65 cm², about 75 mm² and about 60 cm², about 1 cm² and about 80 cm² about 1 cm² and about 75 cm², about 1 cm² and about 70 cm², about 1 cm² and about 65 cm², or about 1 cm² and about 60 cm² in size. In one specific embodiment, the fiber length is between about 10 cm and 50 cm in length, which is particularly useful in the preparation of matrices for implantation.

In one embodiment, the matrix is a single layer of any suitable thickness. In one embodiment, the single layer matrix has a thickness ranging between about 500 μm and about 2 mm thick. In various further embodiments, the single layer thickness may be between about 500 μm and about 1.8 mm, about 500 μm and about 1.5 mm, about 500 μm and about 1.2 mm, about 500 μm and about 1 mm, about 1 mm and about 2 mm, about 1 mm and about 1.8 mm, about 1 mm and about 1.5 mm, about 1.5 mm and about 2 mm, about 1.5 mm and about 1.8 mm, or about 1.8 mm and about 2 mm thick. As will be understood by those of skill in the art, the matrix may contain a plurality (2, 3, 4, 5, 6, 7, 8, 9, 10, or more) layers of matrix (where each individual layer may be the same or differ in one of more characteristics) to produce a three-dimensional scaffold of a desired size. The size will generally be based on the intended use of the matrix; in one embodiment, the multi-layer matrix may be up to about 20 cm in thickness; in various other embodiments, up to about 19 cm, about 18 cm, about 17 cm, about 16 cm, about 15 cm, about 14 cm, about 13 cm, about 12 cm, about 11 cm, about 10 cm, about 9 cm, about 8 cm, about 7 cm, about 6 cm, or about 5 cm in thickness.

The matrices can comprise any embodiment or combination of embodiments of the fibers of the invention. In one embodiment, the plurality of fibers may be predominately (i.e.: 50% or more; such as 60%, 70%, 75%, 80%, 85%, 90%, 95%, 98%, or more) the same diameter/length of fiber. In other embodiments, the matrices may comprise fibers having a variety of diameters and/or lengths as disclosed herein. A broader range of fiber diameters in the matrix results in higher pore dimeters and pore distributions that may favor tissue infiltration of the matrix.

Similarly, the plurality of fibers may predominately comprise the same polyester and/or derivatized cellulose, or may comprise fibers having a variety of different polyester and/or derivatized cellulose components. In one particular embodiment, the matrices predominately comprise PLGA as the polyester and/or comprise CA as the derivatized cellulose component. In other embodiments, the matrices may comprise fibers having predominately the same molecular weight, percentage, and/or ratio of polyester and/or derivatized cellulose component, or may comprise fibers having a variety of polyester and/or derivatized cellulose component molecular weights, percentages, and/or ratios of polyester to derivatized cellulose.

The inventors have discovered that properties of the matrices can be tuned by appropriate manipulation of the cellulose derivative molecular weight and concentration. By way of non-limiting example, use of 30 kD CA present in the fibers at between about 5% to about 15% weight percentage of polyester in the fiber results in matrices comp-rising the fibers having wettability and strength characteristics suitable for repairing soft tissues such as skin, tendon, blood vessel, ligament, cardiac patch, skeletal muscle and nerve scaffolds. At higher CA ratios the matrices comprising the fibers are more of a fibrous gel that allows fluid retention and delivery bioactive molecules. Thus, the resulting matrices can perform all the functions of a hydrogel, but with an ordered structure.

The matrices may comprise any additional components that may be suitable for an intended use of the fibers. Such “cargo” (i.e.: therapeutic agents, diagnostic agents, peptides, growth factors, detectable labels, etc.) may be incorporated into the matrix by inclusion into individual fibers as noted above, or may be bound (covalently or non-covalently) to the matrix using standard techniques in the art.

Matrices comprising such fibers are of particular for soft and hard tissue regeneration, skin applications such as wound healing, and biomedical applications, etc. The cargo can be incorporated into the fiber using standard chemical techniques to link the molecules to the polyester and or the cellulose derivative in the fiber.

In one non-limiting embodiment, the matrices comprise one or more growth factors and/or one or more therapeutics. Such therapeutics may include a therapeutic peptide, for example. By way of non-limiting example, the matrices may comprise an insulin peptide; matrices of this embodiment may be used for delivery of insulin to, for example, modify stem cells into tendon cells for any suitable use, such as rotator cuff regeneration. In other embodiments, the matrices include cargo (such as growth factors) used to stimulate soft tissue regeneration (such as skin, tendon, blood vessel, ligament, cardiac patch, skeletal muscle and nerve scaffolds).

In another embodiment, biological cells are seeded on and/or within the matrix. For various applications, it may be desirable to utilize the matrices to deliver cells to a repair site. Any suitable biological cell can be delivered using the matrices of the invention. In various non-limiting embodiments, the cells are selected from the group consisting of chondrocytes, stem cells (such as mesenchymal stem cells), fibroblasts, keratinocytes, nerve cells, neural progenitors, muscle cells, and muscle progenitors.

In a further aspect, the invention provides methods for making the fiber of any embodiment or combination of embodiments of the invention, comprising

(a) adding a cellulose derivative, such as CA, to a solution of a polyester dissolved in an organic solvent to form a cellulose derivative-polyester solution; and

(b) electrospinning the cellulose derivative-polyester solution to form a cellulose derivative-polyester fiber.

Electrospinning is a simple, elegant and scalable technique to fabricate polymeric fibers. Electrospun nanofiber matrices show morphological similarities to the natural extracellular matrix (ECM), characterized by ultrafine continuous fibers, high surface-to-volume ratio, high porosity and variable pore-size distribution.

Electrospinning is aided by the application of high electric potentials of few kV magnitudes to a pendant droplet of polymer solution/melt from a syringe or capillary tube. By way of example, a polymer jet is ejected from the surface of a charged polymer solution when the applied electric potential overcomes the surface tension. The ejected jet under the influence of applied electrical field travels rapidly to the collector and collects in the form of non-woven web as the jet dries. Various parameters that potentially affect physicochemical properties of the nanofiber matrices during electrospinning include polymer molecular weight, polymer solution properties, applied electrical potential, polymer solution flow rate, distance between spinneret and collector (working distance), motion of the grounded target and ambient parameters (temperature, humidity and air velocity). Fiber diameter, surface morphology, mechanical properties, porosity and pore-size distribution greatly depend on the parameters selected for electrospinning. For instance, increase in viscosity or increase in polymer concentration results in fiber diameter increase. By changing polymer concentration alone it is possible to fabricate the fiber diameters in the range of few nm to several micrometers while keeping other electrospinning parameters constant. Thus, based on the teachings herein, it is well within the level of skill in the art to select appropriate parameters for an intended fiber/matrix according to the present invention.

The cellulose derivative-polyester solution can be prepared via any suitable technique. In one embodiment, the methods may comprise melt-spinning to produce the cellulose derivative-polyester solution. For examples polymer PLGA-CA at definite weight ratios can be heated above their melting point into a solution, and then electrospun to produce fibers.

All embodiments and combinations of embodiments of the fibers of the invention (and the resulting matrices comprising such fibers) can be made using the methods of the present invention. In exemplary embodiments, the cellulose derivative comprises cellulose acetate (CA), ethyl cellulose, ethyl hydroxymethylcellulose, or combinations thereof. In one particular embodiment, the cellulose derivative comprises CA. In one embodiment, the molecular weight of the CA in the fiber is between about 20,000 D and about 75,000 D. In various further embodiments, the CA molecular weight is between about 30,000 D and about 50,000 D, or about 50,000 D. As will be understood by those of skill in the art, use of higher molecular weight CA requires less weight of CA in polyester to produce fibers of similar viscosity to those made with lower molecular weight CA. Thus, the molecular weight of CA present in the fibers can be modified for intended fiber characteristics.

In various embodiments, the polyester is selected from the group consisting of polylactic acid (PLA), polyglycolic acid (PGA), Poly(lactic-co-glycolic acid) (PLGA), poly(caprolactone) (PCL), Polyhydroxyalkanoate (PHA); Polyhydroxybutyrate (PHB); Polyethylene adipate (PEA); Polybutylene succinate (PBS); Poly(3-hydroxybutyrate-co-3-hydroxyvalerate)(PHBV); Polyethylene terephthalate (PET); Polybutylene terephthalate (PBT); Polytrimethylene terephthalate (PTT); Polyethylene naphthalate (PEN), or combinations thereof. In one specific embodiment, the polyester is selected from the group consisting of PLA, PGA, PLGA, PCL) and combinations thereof. In a particular embodiment, the polyester comprises PLGA.

In one particular embodiment, the derivatized cellulose comprises CA and the polyester comprises PLGA. In a further embodiment, the CA is added at between about 5% to about 25% of the weight percentage of polyester (such as PLGA) in the solution.

In further embodiments, the electrospinning is carried out under ambient conditions, with a polymer flow rate of 2-4 mL/hour. In a further embodiment, the solution comprises 15-20% (weight/volume) of the polyester.

While the invention has been described with reference to preferred embodiments, it will be understood by those skilled in the art that various changes may be made and equivalents may be substituted for the elements thereof without departing from the scope of the invention. In addition, many modifications may be made to adapt the teaching of the invention to particular use, application, manufacturing conditions, use conditions, composition, medium, size, and/or materials without departing from the essential scope and spirit of the invention. Therefore, it is intended that the invention not be limited to the particular embodiments and best mode contemplated for carrying out this invention as described herein.

All ranges disclosed herein are inclusive of the endpoints, and the endpoints are independently combinable with each other. Each range disclosed herein constitutes a disclosure of any point or sub-range lying within the disclosed range.

All methods described herein can be performed in any suitable order unless otherwise indicated herein or otherwise clearly contradicted by context. The use of any and all examples, or exemplary language (e.g., “such as”), is intended merely to better illustrate the invention and does not pose a limitation on the scope of the invention or any embodiments unless otherwise claimed.

Chemical compounds are described using standard nomenclature. Unless defined otherwise, all technical and scientific terms used herein have the same meaning as is commonly understood by one of skill in the art to which this invention belongs.

### Example 1

### Materials

Poly(lactic acid-co-glycolic acid) (PLGA), molecular weight Mw=71,000, tetrahydrofuran (THF), acetone and dimethylformamide (DMF) (Fisher Scientific, Atlanta, Ga.) were used for these studies QUANT-IT® PICOGREEN® dsDNA Assay Kit was purchased from Life Technologies. Live/dead cell viability kit was purchased from Molecular Probes (L-3224). Human chondrocytes and cell culture media (Eagles Minimum Essential Medium (EMEM)) were obtained from ATCC (Manassas, Va.). Fetal bovine serum (FBS), antibiotics (Penicillin, Streptomycin P/S) and trypsin-EDTA, were purchased from Sigma (St. Louis, Mo.).

### Fabrication of PLGA-CA-Fiber Matrices

Fiber matrices were fabricated using a conventional electrospinning setup reported earlier. The apparatus consists of a 10 mL glass syringe fitted with a 20 gauge blunt end needle and a grounded electrode covered with an aluminum foil sheet. PLGA of molecular weight was dissolved in an organic solvent mixture of THF:DMF (3:1 ratio) at varying concentrations. To this a calculated amount of CA molecular weight 50,000 was added as a dopant and its concentration varied between 5-20% (wt. % of PLGA) and allowed to dissolve. Polymer solution flow was adjusted using a programmable syringe pump (Kent Scientific Corporation USA) to a flow rate of 2 mL/h. A Gamma High Voltage Supply ES40P-20W (0-40 kV, 20 W, Gamma High Voltage Research) with a low current output was used to maintain a potential gradient of 1 kV/cm. Electrospinning was carried out at ambient temperature and pressure. The spun fiber matrices were dried under vacuum at room temperature for 24 h.

### Matrix Characterization

Fiber matrices were characterized for material interaction, thermal properties and surface morphologies using a variety of analytical techniques. Fiber matrices were characterized using Fourier Transform Infrared (FTIR) Spectroscopy (ThermoScientific Nicolet iS10) was used to study possible interaction between two polymer components in the mixture. Nuclear magnetic resonance (NMR) Spectroscopy (Bruker DMX 500 MHz NMR) was used to study possible interaction between two polymer components in the mixture. Transmission electron microscopy (TEM) was used to study possible interaction between two polymer components in the fiber matrix. Matrices were characterized using Differential Scanning Calorimetry (DSC) using a TA Instruments Q100 to study the changes in thermal properties of the matrix. The morphologies of the fiber matrices were characterized by scanning electron microscopy (SEM) (JEOL JSM 6335F) and matrices were coated with Au/Pd using a HUMMER® V sputtering system (Technics Inc., Baltimore, Md.) before viewing with SEM.

### Incubation in Physiological pH and Temperature

Fiber matrices were cut into circular discs using cork-borer no. 10 with an area of approximately 2.27 cm² and a thickness of 0.38-0.42 mm. These circular discs were incubated in phosphate buffered saline (PBS) pH-7.4 at 37° C. in an incubator. The gross morphological changes were observed at various time points of 4, 12, and 24 hours following incubation. These fiber matrices characterized for changes in morphology (optical micrographs and SEM) and mechanical properties. Fiber matrices measuring 5×10 mm (dog bone shaped) were incubated in PBS overnight at 37° C. in an incubator prior to testing them for tensile properties. An Instron 5544 Compression/Tension testing apparatus was used in tensile testing mode with 10 mm/min with a 50N load cell (n=6).

### In Vitro Cell Culture

Circularly cut fiber matrices were soaked in 70% ethanol for 20 min, and then dried and sterilized under UV light for 1 h on each side prior to cell seeding. Each matrix was seeded with 50,000 cells with human articular chondrocytes and 1.8 mL of growth media was added to the samples and then changed completely every other day. Cell proliferation and viability was evaluated at various time points of 3, 7, 14 and 21 days.

### Chondrocyte Viability

Chondrocyte viability on fiber matrices were imaged with a live/dead cell viability kit (Molecular Probes, L-3224). In brief, calcein AM enters live cells and reacts with intracellular esterase to produce a bright green fluorescence, while ethidium homodimer-1 enters only dead cells with damaged membranes and produces a bright red fluorescence upon binding to nucleic acids. Fiber matrices were imaged on 3, 7, 14 and 21 days using a BioRad Radiance 2100 Multiphoton/Laser Scanning Confocal Microscope (LSCM) at different magnifications.

### Results

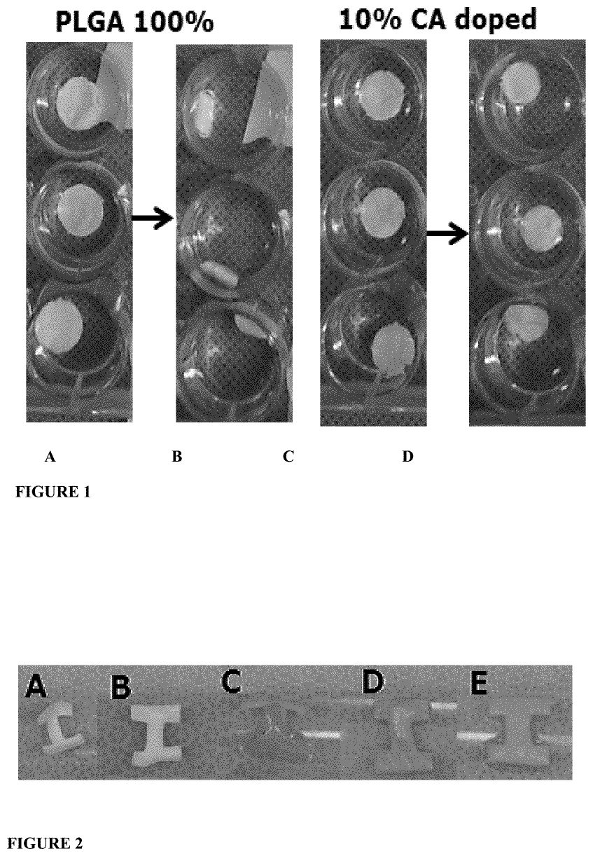

FIG. 1 shows the gross morphology of electrospun neat PLGA (left) and with 10% doping of CA (right) before and after seven days of incubation at 37° C. in PBS (n=3). There is a noticeable loss of scaffold form and structure in the 100% PLGA in comparison to the 10% CA doped form.

FIG. 2 shows the morphology of fiber matrices doped with varying amount of CA following incubation in PBS at 37° C. for tensile testing. All these matrices were cut into dog-bone shapes measuring 5×10 mm (1:2 ratio). A through E represent increasing amounts of cellulose acetate doping, 0% to 20%. (A—PLGA neat, B—with 5% CA, C—with 10% CA, D—with 15% CA, E—with 20% CA). With an increase in CA content, the matrix tends to absorb more water (hydrophilic) and loses brittleness.

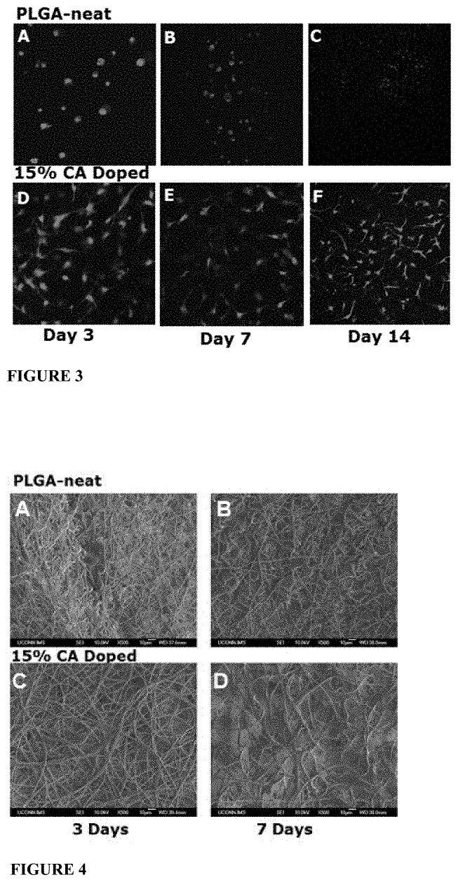

FIG. 3 shows cell survival and morphology of human articular chondrocytes seeded on neat electrospun PLGA and 15% CA doped fiber matrices were determined by viability/cytotoxicity assay. Live cells appear as fluorescent green color and dead cells as fluorescent red. A noticeable change in cellular morphology and proliferative performance is seen. (Images A, B, D, and E—are 20×. Images C and F are 10×.)

FIG. 4 shows SEM micrographs of human articular chondrocytes seeded fiber matrices at a time point of 3 and 7 days on neat PLGA and 15% CA doped fiber matrices. Similar cell spreading morphology was seen on 15% CA doped fiber matrices as compared to neat PLGA. Neat PLGA fiber matrices present alterations in fiber morphology, becoming more tortuous in nature.

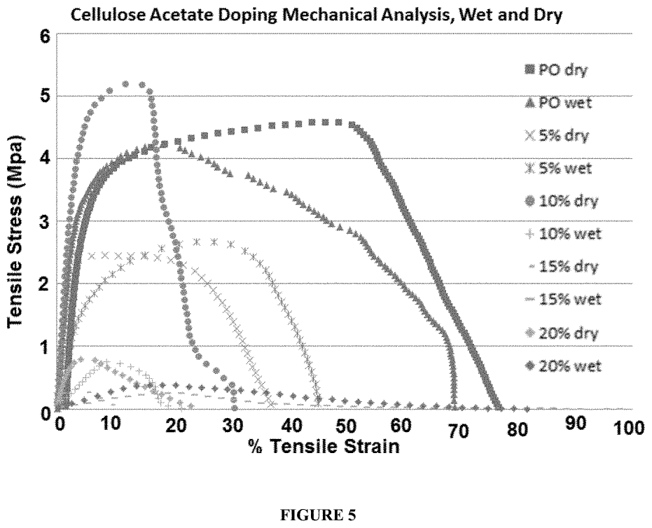

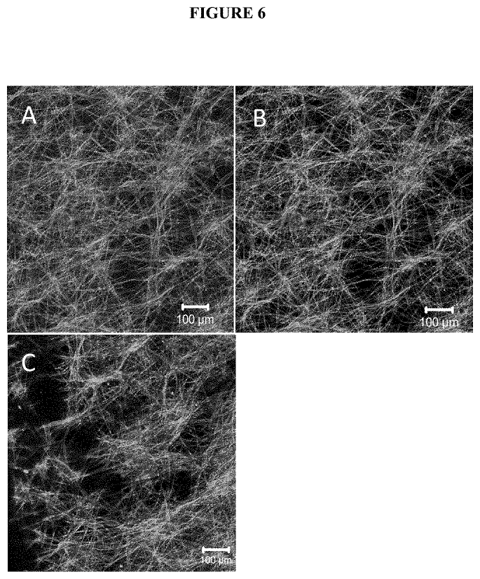

FIG. 5 is a graph showing the relative tensile modulus (stress vs. strain) results of cellulose acetate doped PLGA scaffolds after dry and wet (37° C. buffered media) incubation. The PO (PLGA Only) loss in area under the curve after physiological incubation provides mechanical data supporting the increasingly stiff and brittle nature of the pure PLGA scaffold following exposure to physiological environments. CA doped matrices appear to soften following exposure with greater amounts correlating directly with CA content.

FIG. 6 provides confocal images showing the feasibility of uniform model protein functionalization on the fiber matrix PLGA: CA (80:20) with BSA. For visualization FITC conjugated BSA (BSA-FITC) was chemically tethered to fiber matrix via hydroxyl groups present on CA nanofibers. Prior to imaging matrices were washed repeatedly with DI water to remove any adsorbed protein and incubated in PBS for 2 hours. These images were taken at the different locations of the matrix show uniformity of the matrix. Magnification 10×.

We have also tested this approach with poly(caprolactone) (PCL) and uniform BSA functionalization was achieved, and CA addition improved PCL matrix hydrophilicity.

As demonstrated by these results, PLAGA-CA electrospun mixtures containing 5%, 10%, 15% and 20% CA (30K) showed enhanced morphological performance during in vitro analysis as seen through gross examination after incubation in PBS at physiological temperatures at times as early as 4-24 hours. These results were further supported by analysis through scanning electron microscopy shown by maintenance of original micro-nanofiber scaffold morphology at said time points. Manipulation of scaffolds following prolonged physiological incubation suggests maintenance of mechanical characteristics relevant to improved soft tissue mimicry such as pliability, and elasticity. Mechanical evaluation of incubated scaffolds depicted the expected maintenance of scaffold pliability through the display of a more elastic deformation to failure in comparison to PLAGA alone. Cytocompatibility of the electrospun mixtures by SEM analysis and fluorescent Live/Dead staining showed increased cellular proliferation, cell attachment and altered morphology, presumably due to altered scaffold topography, ductility, and hydrophilicity.

We claim:

1. A fiber, wherein the fiber has a diameter of less than about 5000 nm, wherein the fiber comprises
(a) Poly(lactic-co-glycolic acid) (PLGA) having a molecular weight between about 40 kD and about 250 kD, wherein the PLGA is at least 70% by weight of the fiber; and
(b) cellulose acetate (CA) having a molecular weight between about 20,000 D and about 75,000 D, wherein the CA is between 5% to 20% by weight of the fiber.

2. The fiber of claim 1, wherein the CA is at between 10% to 20% by weight of the fiber.

3. The fiber of claim 1, wherein the CA is at between 5% and 15% by weight of the fiber.

4. The fiber of claim 1, wherein the fiber has a diameter between about 100 nm and about 1500 nm.

5. The fiber of claim 1, further comprising between about 1% to about 15% by weight of a cargo.

6. The fiber of claim 5, wherein the cargo is selected from the group consisting of therapeutic agents, diagnostic agents, peptides, growth factors, and detectable labels.

7. A matrix comprising a plurality of the fibers of claim 1, wherein the fibers are linked together to form the matrix.

8. The matrix of claim 7, wherein the matrix is between about 50 mm² and about 80 cm² in size.

9. The matrix of claim 7, wherein the matrix comprises a fiber layer that is between about 500 μm and about 2 mm thick.

10. The matrix of claim 9, wherein the matrix comprises a plurality of fiber layers.

11. The matrix of claim 7, comprising biological cells seeded on and/or within the matrix.

12. The matrix of claim 7, further comprising a cargo loaded on and/or within the matrix.

13. A method for making a fiber, comprising:
(a) adding a cellulose derivative, such as CA, to a solution of a polyester dissolved in an organic solvent to form a cellulose derivative-polyester solution; and
(b) electrospinning the cellulose derivative-polyester solution to form a cellulose derivative-polyester fiber.
